(19) United States
(12) Patent Application Publication
(10) Pub. No.: US 2005/0245617 A1
(43) Pub. Date: Nov. 3, 2005

(54) Methods and compositions for the treatment of CNS-related conditions

(76) Inventors: Laurence R. Meyerson, Carlsbad, CA (US); Gregory T. Went, Mill Valley, CA (US); Timothy J. Fultz, Pleasant Hill, CA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/048,002

(22) Filed: Jan. 31, 2005

Related U.S. Application Data
(60) Provisional application No. 60/540,713, filed on Jan. 29, 2004.
(60) Provisional application No. 60/544,838, filed on Feb. 13, 2004.

(51) Int. Cl. A61K 31/137; A61K 31/13

(52) U.S. Cl. 514/649; 514/662

(57) ABSTRACT

The invention provides methods and compositions for the treatment of dementia-related conditions, such as Parkinson's disease and Alzheimer's disease.

### RELATED APPLICATION

This application claims priority to U.S. Ser. No. 60/540,713, filed Jan. 29, 2004, and No. 60/544,838, filed Feb. 13, 2004. The contents of these applications are incorporated herein by reference in their entirety.

### FIELD OF THE INVENTION

This invention relates to compositions and methods for treating CNS-related conditions, such as Parkinson's disease and Alzheimer's disease.

### BACKGROUND OF THE INVENTION

Monoamine oxidase inhibitors (MAOi, A or B) are used in the clinic for the symptomatic treatment of a number of neurological and neuropsychiatric disorders, including early Parkinson's disease (PD) depression, and bipolar depression. Their benefit has been attributed to both the inhibitory action on the enzymatic degradation of amines (e.g., dopamine, serotonin, tyramine and 2-phenylethylamine) as well a poorly understood free-radical scavenging activity. Recently, this secondary action has been reported to be associated with the antagonism of GAPDH mediated apoptosis. GAPDH is apparently found translocated into the nucleus of apoptotic cells and the nuclear levels are associated with numerous diseases including Parkinson's, Alzheimer's and Huntington's diseases. The administration of MAO inhibitors, however, is associated with a number of debilitating side effects that limit their use. These effects include, for example, nausea, dizziness, lightheadedness, fainting, abdominal pain, confusion, hallucinations, dry mouth, vivid dreams, dyskinesias, and headache.

Thus, there is a clear need to find therapeutic modalities that would maintain or improve the therapeutic benefits of MAO inhibitors (MAOi) and other compounds that antagonize GAPDH mediated apoptosis (GAPDHai) while reducing or eliminating such undesirable side effects.

### SUMMARY OF THE INVENTION

In general, the present invention provides methods and compositions for treating CNS-related conditions, such as Parkinson's disease and Alzheimer's disease, by administering to a subject in need thereof a combination of an NMDA receptor antagonist and a MAO inhibitor (refered to as “MAOi”) or an antagonists of GAPDH mediated apoptosis (termed “GAPDHai, (e.g., selegiline and rasagiline). The administration of the combinations described herein results in the alleviation and prevention of symptoms associated with or arising from CNS-related conditions or dementia including, for example, loss of memory, loss of balance, hallucinations, depression, delusions, agitation, withdrawal, depression, communication problems, cognitive loss, personality change, confusion, and insomnia.

The NMDA receptor antagonist, the MAO inhibitor or GAPDHai, or both agents may be provided in a controlled, extended release form with or without an immediate release component in order to maximize the therapeutic benefit while reducing unwanted side effects. Taken together, a formulation of this type yields a more stable Cratio as a function of time, where Cratio is defined as the measured concentration ratio between the two active components. When referring to an agent, the term “C” designates the concentration of such agent in a patient sample (e.g. blood, serum, cerebrospinal fluid) at any point in time. Thus, the “Cmean” of an agent refers to the mean concentration of such agent in the patient sample as measured by any standard assay method known in the art over a set period of time. The “Cmax” of an agent refers to the maximum concentration typically measured for such agent at any point in time within a defined range. Taken together, a formulation of this type yields a more stable Cratio as a function of time, where Cratio is defined as the measured concentration ratio between the two active components. Thus, the relative Cratio of the NMDA receptor antagonist and MAO inhibitor or GAPDHai may be 0.4-2.5.

In a preferred embodiment of the present invention, less than 50% of the NMDA receptor antagonist, the MAO or GAPDHai, or both have been transported into the circulatory or neural system within one hour of such administration. The pharmaceutical composition may be formulated for oral, topical transepithelial, subdermal, intravenous, intranasal, or inhalation delivery. Optionally, the pharmaceutical composition may be formulated as a suspension, capsule, tablet, suppository, lotion, patch, or device (e.g., a subdermally implantable delivery device or an inhalation pump).

Although any non-toxic NMDA receptor antagonist is useful for the methods and compositions of the invention, low and even moderate affinity NMDA receptor antagonists (see, for example, Parsons et al., Neuropharmacology 34:1239-58, 1995) are preferred. Such NMDA receptor antagonists are typically less toxic than high affinity NMDA receptor antagonists, which may exhibit psychotropic side-effects at or near therapeutic doses. Thus, the NMDA receptor antagonist may be, for example, an aminoadamantine derivative including memantine (1-amino-3,5-dimethyladamantane), rimantadine (1-(1-aminoethyl)adamantane), or amantadine (1-amino-adamantane). The MAO inhibitor or GAPDHai are to be taken from class of drugs that have been shown to inihibit apoptosis, including those that are presumed to act as MOA inhibitors, free radical scavengers or exhibit inhibition of GAPDH mediated apoptosis (see, for example, Chuang et al., Annual Review of Pharmacology and Toxicology, 45:269-290, 2004), including L-deprenyl/SELEGILINE™, desmethyldeprenyl, N-propargyl-1 (R)-aminoindan/Rasagaline™, phenelzine/NARDIL™, tranycypromine/PARNATE™, CGP3466, Furazolidone, Isocarboxazid/MARPLAN (Oxford Pharm), Pargyline HCl, Pargyline HCl and methyclothiazide, and Procarbazine HCl/Matulane (Sigma Tau). The present invention differs from prior studies by providing dose optimization or release modifications to reduce adverse effects associated with each agent.

In some embodiments, the amount of the NMDA receptor antagonist administered to a subject may be equal to, or less than the amount of NMDA receptor antagonist typically administered to subjects. For example, the amount of memantine required to positively affect the patient response (inclusive of adverse effects) may be 2.5-40 mg per day rather than the typical 10-20 mg per day administered without the extended release or MAOi or GAPDHai activity. Similarly, in some embodiments the amount of the MAOi or GAPDHai administered to the subject is less than the amount of than that administered to the subject to obtain the same therapeutic effect for treating CNS-related conditions observed when the MAOi or GAPDHai is administered in the absence of a controlled or modified release and the NMDA receptor antagonist. Of course, in some combinations lowered amounts of both the NMDA receptor antagonist and the MAOi or GAPDHai are administered in a unit dose relative to the amount of each administered in the absence of the other with similar or improved patient response. Such a response may be additive or synergistic, as described below.

In some embodiments, higher doses of the MAOi or GAPDHai are administered to the subject relative to the amount of the MAOi or GAPDHai that could be administered in the absence of controlling the release, mode of administration and the NMDA receptor antagonist. In some embodiments, higher doses of the NMDA receptor antagonist are administered to the subject relative to the amount of the NMDA receptor antagonist that could be administered in the absence of controlling the release, mode of administration and the or GAPDHai. In a preferred embodiment, the NMDA antagonist and the MAOi or GAPDHai may be admixed in a single composition and delivered in an oral, patch or transnasal formulation.

Alternatively, the two agents are delivered in separate formulations sequentially, or within one hour, two hours, three hours, six hours, 12 hours, or 24 hours of each other. If administered separately, the two agents may be administered by the same or different routes of administration three times a day, twice a day, once a day, or even once every two days.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present Specification, including definitions, will control. In addition, the materials, methods, and examples are illustrative only and not intended to be limiting. All parts and percentages are by weight unless otherwise specified.

Other features and advantages of the invention will be apparent from the following detailed description and claims.

### BRIEF DESCRIPTION OF THE FIGURES

FIG. 1 is a graph showing that controlled release of the NMDA receptor antagonist results in a reduction in dC/dt.

FIGS. 2A-2C is a series of graphs showing the release profiles and Cratio for controlled release combination product.

FIGS. 3A and 3B are graphs comparing the anticipated 12 hour controlled release with the anticipated 24 hour controlled release.

### DETAILED DESCRIPTION OF THE INVENTION

The present invention provides methods and compositions for treating or preventing CNS-related conditions, such as Parkinson's disease and Alzheimer's disease. The combination includes a first component that is an NMDA receptor antagonist and a second component that is a MAO inhibitor or GAPDH mediated apoptosis inhibitor. The combination is administered such that symptoms are alleviated or prevented, or alternatively, such that progression of the CNS-related condition is reduced. Desirably, either of these two agents, or even both agents, is formulated for extended release, thereby providing a concentration and optimal concentration ratio over a desired time period that is high enough to be therapeutically effective but low enough to avoid adverse events associated with excessive levels of either component in the subject.

Role of Glutamate in Neurological Disorders

Excitatory amino acid receptors are the primary mediators of excitatory synaptic transmissions (i.e., stimulation of neurons) in the brain, participating in wide-ranging aspects of both normal and abnormal central nervous system (CNS) function. The principle excitatory receptor, the N-Methyl-D-Aspartate (NMDA) receptor and its associated calcium (Ca2+) permeable ion channel are activated by glutamate and its co-agonist glycine. NMDA receptor activity and consequent Ca2+ influx are necessary for long-term potentiation (a correlate of learning and memory).

Aberrant glutamate receptor activity has been implicated in a large number of neurodegenerative conditions. In this regard, the abnormal activation of the NMDA receptor that may result from elevated levels of glutamate, for example, can lead to sustained activity of the receptor's ion channel (often lasting for minutes rather than milliseconds), thereby allowing Ca2+ to build-up. The excessive influx of Ca2+ eventually leads to an increase in intracellular reactive NO, increased free radical concentrations, resulting degradation in cell-cell communication, extended release of excitatory amino acids, and inappropriate stimulation of adjacent neurons, and ultimately, cell death (apoptosis). Thus, strategies that reduce glutamate-mediated excitotoxicity are needed, particularly those that inhibit the consequences of over-stimulation while preserving normal glutamate activity.

NMDA Receptor Antagonists

Certain NMDA receptor antagonists have the ability to attenuate the effects of elevated glutamate without adversely affecting normal glutamatergic activity in the brain. Most of these are termed uncompetitive antagonists owing to their interaction with the Ca2+ channel in its open state. The safest of these (e.g., memantine) act in a manner to block and leave the channel quickly. These drugs have excellent systemic safety profiles, with a fairly narrow range of activity.

MOA Inhibitors and GAPDHai

Certain drugs that are known to modulate MOA activity, as well as others that have demonstrated inhibition of apoptosis via free radical scavenging or GAPDH mediated apoptosis inhibition are the subject of this invention. One such member of this class is deprenyl/Selegiline™ which is thought to act by inhibiting the generation of free-radicals in at-risk neurons to decrease the oxidative burden and hence lower the risk of apoptosis, and by blocking the transport of GAPDH into the nucleus where it accelerates apoptosis. These drugs display excellent activity profiles, but are limited by toxicity and food interactions which limit their use. Other drugs which are the subject of this invention due to their apparent GAPDH modulatory effects anti-sense oligonucleotides and RNAi oligonucleotides.

Unique Combination Effect

One aspect of this invention is to formulate these agents in a manner in which the combined activity benefit is sufficient to allow for the reduction in the adverse events. The optimum ratio of components in this case results from the novel synergy between the mechanisms of action of these drugs. Certain NMDA receptor antagonists are effective at blocking excessive Ca2+, thereby reducing apoptosis presumably through a reduction in intracellular free radical damage and possible reduced effects on intracellular reaction NO species. We have discovered a mechanism by which certain MAO inhibitors and GAPDHais can act synergistically with certain NMDA receptor antagonists to reduce the intracellular effects of Ca2+. These MAO or GAPDH mediated apoptosis inhibitors inhibit the transport/translocation of GAPDH from the cytoplasm across the nuclear membrane into the nucleus. Thus, a combination of the present invention allows for direct intervention at two-points in the same biological pathway, which will have an unanticipated and synergistic benefit in the patient.

The amounts and ratios of the NMDA receptor antagonist and the MAO inhibitor or GAPDHai can be varied to maximize the therapeutic benefit and minimize the toxic or safety concerns. In one example, the NMDA receptor antagonist can range from 20% to 100% of its normal effective dose and the MAO inhibitor or GAPDHai can range from 20% to 100% of its normal effective dose. The precise ratio may vary by the condition being treated. In one example, the amount of memantine can range from 2.5 to 40 mg per day, and the amount of 1-deprenyl from 1 to 10 mg/day.

Formulation Benefits

Certain NMDA receptor antagonists, such as memantine, readily cross the blood-brain barrier, achieving similar concentrations in the extra cellular fluid surrounding brain tissue and systemic serum. Ideally, the NMDA receptor antagonist should be present at a concentration sufficient to reduce the symptoms of the disease in the absence of debilitating side effects. In the present dosage forms however, these drugs, some of which have a relatively long half-life, require an initial dose escalation or “titration” to avoid side effects associated with initial exposure. This leads to difficulty in achieving adequate patient compliance, which is further exacerbated by the complicated dosing schedules of therapeutic modalities used for neurological or neuropyschiatric disorders.

Control of drug release is therefore particularly desirable for reducing and delaying the peak plasma level without affecting the extent of drug availability. Therapeutic levels are achieved while minimizing debilitating side-effects that are usually associated with immediate release formulations. Furthermore, as a result of the delay in the time to obtain peak plasma level and the potentially extended period of time at the therapeutically effective plasma level, the dosage frequency may be reduced to, for example, once or twice daily dosage, thereby improving patient compliance and adherence.

Accordingly, the combination of the invention allows the NMDA receptor antagonist and the MAO inhibitor or GAPDHai to be administered in a combination that improves efficacy and avoids undesirable side effects of both drugs. For example, side effects including psychosis and cognitive deficits associated with the administration of NMDA receptor antagonists may be lessened in severity and frequency through the use of controlled-release methods and the synergy of the combination therapy, both aspects of the present invention. Also, side effects associated with the use of MAO inhibitor or GAPDHai may be reduced in severity and frequency through controlled release and the synergy of the combination therapy as previously noted. Furthermore, controlled-release of the active pharmaceutical ingredients of the formulation enables the achievement of desired Cmax/Cmean profiles during the course of administration and the maintenance of an optimal concentration ratio of the active components throughout the course of treatment.

Modes of Administration

The combination of the invention may be administered in either a local or systemic manner or in a depot or sustained release fashion. In a preferred embodiment, the NMDA receptor antagonist, the MAO inhibitor or GAPDHai, or both agents may be formulated to provide controlled, extended release (as described herein). For example, a pharmaceutical composition that provides controlled release of the NMDA receptor antagonist, the MAO inhibitor or GAPDHai, or both may be prepared by combining the desired agent or agents with one or more additional ingredients that, when administered to a subject, causes the respective agent or agents to be released at a targeted rate for a specified period of time. These agents may be delivered preferably in an oral, transdermal or intranasal form.

The two components are preferably administered in a manner that provides the desired effect from the first and second components in the combination. Optionally, the first and second agents are admixed into a single formulation before they are introduced into a subject. The combination may be conveniently sub-divided in unit doses containing appropriate quantities of the first and second agents. The unit dosage form may be, for example, a capsule or tablet itself or it can be an appropriate number of such compositions in package form. The quantity of the active ingredients in the unit dosage forms may be varied or adjusted according to the particular need of the condition being treated.

Alternatively, the NMDA receptor antagonist and the MAO inhibitor or GAPDHai of the combination may not be mixed until after they are introduced into the subject. Thus, the term “combination” encompasses embodiments where the NMDA receptor antagonist and the MAO inhibitor or GAPDHai are provided in separate formulations and are administered sequentially. For example, the NMDA receptor antagonist and the MAO inhibitor or GAPDHai may be administered to the subject separately within 2 days, 1 day, 18 hours, 12 hours, one hour, a half hour, 15 minutes, or less of each other. Each agent may be provided in multiple, single capsules or tablets that are administered separately to the subject. Alternatively, the NMDA receptor antagonist and the MAO inhibitor or GAPDHai are separated from each other in a pharmaceutical composition such that they are not mixed until after the pharmaceutical composition has been introduced into the subject. The mixing may occur just prior to administration to the subject or well in advance of administering the combination to the subject.

If desired, the NMDA receptor antagonist and the MAO inhibitor or GAPDHai may be administered to the subject in association with other therapeutic modalities, e.g., drug, surgical, or other interventional treatment regimens. Where the combination includes a non-drug treatment, the non-drug treatment may be conducted at any suitable time so long as a beneficial effect from the co-action of the combination and the other therapeutic modalities is achieved. For example, in appropriate cases, the beneficial effect is still achieved when the non-drug treatment is temporally removed from the administration of the therapeutic agents, perhaps by days or even weeks.

NMDA Receptor Antagonist Component

In general, any non-toxic NMDA receptor antagonist is useful for the methods and compositions of the invention so long as it is non-toxic when used in the composition. The term “nontoxic” is used in a relative sense and is intended to designate any substance that has been approved by the United States Food and Drug Administration (“FDA”) for administration to humans or, in keeping with established regulatory criteria and practice, is susceptible to approval by the FDA for administration to humans.

The NMDA receptor antagonist is desirably an amino adamantane compound. Suitable amino adamantane compounds are well known in the art and include, for example, memantine (1-amino-3,5-dimethyladamantane), rimantadine (1-(1-aminoethyl)adamantane), amantadine (1-amino-adamantane), as well as pharmaceutically acceptable salts thereof. Memantine is described, for example, in U.S. Pat. Nos. 3,391,142, 5,891,885, 5,919,826, and 6,187,338. Amantadine is described, for example, in U.S. Pat. Nos. 3,152,180, 5,891,885, 5,919,826, and 6,187,338. Additional aminoadamantane compounds are described, for example, in U.S. Pat. Nos. 4,346,112, 5,061,703, 5,334,618, 6,444,702, 6,620,845, and 6,662,845. All of these patents are hereby incorporated by reference.

If desired, the NMDA receptor antagonist may include one or more aminoadamantane compounds that are non-toxic when used as part of the combination. Accordingly, the aminoadamantane compound or compounds are non-toxic when used with the second agent of the combination even though levels of the aminoamantane compound or compounds may otherwise be toxic if administered to the subject in the absence of the second agent of the combination. The term “nontoxic” is used in a relative sense and is intended to designate any substance that has been approved by the United States Food and Drug Administration (“FDA”) for administration to humans or, in keeping with established regulatory criteria and practice, is susceptible to approval by the FDA for administration to humans.

Further NMDA receptor antagonists include, for example, ketamine, eliprodil, ifenprodil, dizocilpine, remacemide, iamotrigine, riluzole, aptiganel, phencyclidine, flupirtine, celfotel, felbamate, spermine, spermidine, levemopamil, dextromethorphan ((+)-3-hydroxy-N-methylmorphinan) and its metabolite, dextrorphan ((+)-3-hydroxy-N-methylmorphinan)a pharmaceutically acceptable salt or ester thereof, or a metabolic precursor of any of the foregoing.

The NMDA receptor antagonist may be provided so that it is released at Cₘₐₓ/Cₘₑₐₙ of approximately 2 or less for approximately 2 hours to at least 8 hours after the NMDA receptor antagonist is introduced into a subject. The pharmaceutical composition may be formulated to provide memantine in an amount shown in Example 4, between 1 and 80 mg/day, 5 and 40 mg/day, or 10 and 20 mg/day; amantadine in an amount ranging between 25 and 500 mg/day, 25 and 300 mg/day, or 100 and 300 mg/day; dextromethorphan in an amount ranging between 1′-5000 mg/day, 1-1000 mg/day, and 100-800 mg/day, or 200-500 mg/day. Pediatric doses will be lower than those determined for adults.

TABLE 1
Pharmacokinetics in humans and rats for slected NMDAr antagonists

| Compound | Human PK | References |
|---|---|---|
| Memantine | 56 hrs | Namenda NDA submission 21-487 |
| Rimantadine | 25 hrs | Chladek et al. In. J. Clin Pharm 39: 179-184 |
| Amantadine | 16 hrs | Aoki, et al. Clin Pharm. 26: 729-736 (1979) |

Second Agent Component: MAO Inhibitor or GAPDH Mediated Apoptosis Inhibitors

Suitable MAO inhibitors or GAPDHai include, for example, L-deprenyl/SELEGILINE™, desmethyldeprenyl, N-propargyl-1(R)-aminoindan/Rasagaline™, desmethlydeprenyl, phenelzine/NARDIL™, tranycypromine/PARNATE™, CGP3466, Furazolidone, Isocarboxazid/MARPLAN (Oxford Pharm), Pargyline HCl, Pargyline HCl and methyclothiazide, and Procarbazine HCl/Matulane (Sigma Tau). [TF insert list from far above], antisense or RNAis of GAPDH.

Doses of the MAO inhibitor or GAPDHai in the combination depends on the specific agent used, as shown in Example 4 below, typically range between 1 mg/day to about 200 mg/day. For example, doses of L-deprenyl in the combination may range between 1 and 10 mg/day in adults whereas that of Rasagiline may range from 1 to 20 mg/day. Ani-apoptotic doses may be much lower than those typically used. Pediatric doses will be lower than those determined for adults.

TABLE 2
Pharmacokinetics in humans and rats for selected MAO
inhibitors/GAPDHais

| Compound | Human PK | References |
|---|---|---|
| Deprenyl/Selegiline | 1.5-8.6 hrs | Barret et al., Am. J. of Ther., 4: |
| | | 298-313, 1996 |
| Desmethyldeprenyl | 3.8-9.5 hrs | Barret et al., Am. J. of Ther., 4: |
| | | 298-313, 1996 |
| N-propargyl-1(R)- | 1.8 hrs | Stern et al., Movement |
| aminoindan/Rasageline | | Disorders, 19: 916-923, 2004 |

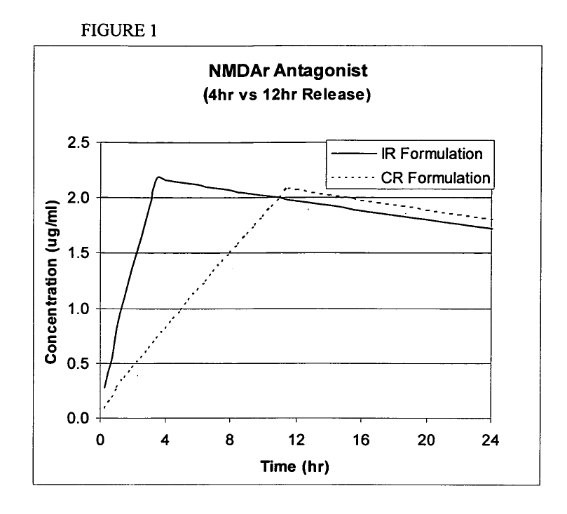

In a representative example, at least 50% of the NMDA receptor antagonist is provided in an extended release dosage form and upon the administration to a subject (e.g., a mammal such as a human), the NMDA receptor antagonist has a Cₘₐₓ/Cₘₑₐₙ of approximately 1.5 from about 2 hours to approximately 8 hours or longer following administration to a subject. If desired, the release of the NMDA receptor antagonist may be monophasic or multiphasic (e.g., biphasic). Moreover, the MAO or GAPDHai may be formulated as an extended release composition, having a Cₘₐₓ/Cₘₑₐₙ of approximately 2 from about 2 hours to approximately 8 hours or longer following administration to a subject. In addition, the controlled release formulation leads to an initial concentration slope (dC/dt) less than that for an immediate release formulation, preferably less than 50% of the immediate release form (see FIG. 1).

Optimal Ratios of Components

In addition to the specific combinations disclosed herein, combinations made of a first aminoadamantane compound and a MAO inhibitor or GAPDHai may be identified by testing the ability of a test combination of a selected aminoadamantane compound and one or more MAO inhibitor or GAPDHai to lessen the symptoms of dementia-related conditions (e.g., Parkinson's disease and Alzheimer's disease). An embodiment for selecting this ratio is described in Example 1, in which the optimal synergistic ratio of the two components is estimated from in vitro neuronal assays, or in Example 2, from in vivo models. Preferred combinations are those in which either raise the beneficial effect or achieve a lower therapeutically effective amount of the NMDA receptor antagonist and/or MAO inhibitor or GAPDHai relative to the same amount of the NMDA receptor antagonist and/or MAO inhibitor or GAPDHai required to obtain the same effect when each agent is tested separately. By beneficial effect here we mean an increase in the effectiveness toward the disease or symptoms and/or a decrease in the adverse effects.

As for every drug, the dosage is an important part of the success of the treatment and the health of the patient. In every case, in the specified range, the physician has to determine the best dosage for a given patient, according to his sex, age, weight, pathological state and other parameters. In some cases, it may be necessary to use dosages outside of the ranges stated in pharmaceutical packaging insert to treat a subject. Those cases will be apparent to the prescribing physician or veterinarian.

Formulations for Specific Routes of Administration

Combinations can be provided as pharmaceutical compositions that are optimized for particular types of delivery. For example, pharmaceutical compositions for oral delivery are formulated using pharmaceutically acceptable carriers that are well known in the art. The carriers enable the agents in the combination to be formulated, for example, as a tablet, pill, capsule, solution, suspension, sustained release formulation; powder, liquid or gel for oral ingestion by the subject.

Alternatively, the compositions of the present invention may be administered transdermally via a number of strategies, including those described in U.S. Pat. Nos. 5,186,938, 6,183,770, 4,861,800 and WO 89/09051. The benefits of patching the present composition is the fact that both molecules have relatively high skin fluxes, and the adverse events and pharmacokinetic variability associated with first pass metabolism of MAO inhibitors, including deprenyl/selegiline™.

Pharmaceutical compositions containing the NMDA receptor antagonist and/or second agent of the combination can also be delivered in an aerosol spray preparation from a pressurized pack, a nebulizer or from a dry powder inhaler. Suitable propellants that can be used in a nebulizer include, for example, dichlorodifluoro-methane, trichlorofluoromethane, dichlorotetrafluoroethane and carbon dioxide. The dosage can be determined by providing a valve to deliver a regulated amount of the compound in the case of a pressurized aerosol.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. Preferably the compositions are administered by the oral, intranasal or respiratory route for local or systemic effect. Compositions in preferably sterile pharmaceutically acceptable solvents may be nebulized by use of inert gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices that deliver the formulation in an appropriate manner.

In some embodiments, for example, the composition may be delivered intranasally to the cribriform plate rather than by inhalation to enable transfer of the active agents through the olfactory passages into the CNS and reducing the systemic administration. Devices commonly used for this route of administration are included in U.S. Pat. No. 6,715,485. Compositions delivered via this route may enable increased CNS dosing or reduced total body burden reducing systemic toxicity risks associated with certain drugs.

Additional formulations suitable for other modes of administration include rectal capsules or suppositories. For suppositories, traditional binders and carriers may include, for example, polyalkylene glycols or triglycerides; such suppositories may be formed from mixtures containing the active ingredient in the range of 0.5% to 10%, preferably 1%-2%.

The combination may optionally be formulated for delivery in a vessel that provides for continuous long-term delivery, e.g., for delivery up to 30 days, 60 days, 90 days, 180 days, or one year. For example the vessel can be provided in a biocompatible material such as titanium. Long-term delivery formulations are particularly useful in subjects with chronic conditions, for assuring improved patient compliance, and for enhancing the stability of the combinations. Formulations for continuous long-term delivery are provided in, e.g., U.S. Pat. Nos. 6,797,283; 6,764,697; 6,635,268, and 6,648,083.

If desired, the components may be provided in a kit. The kit can additionally include instructions for using the kit. In some embodiments, the kit includes in one or more containers the NMDA receptor antagonist and, separately, in one or more containers, the MAO inhibitor or GAPDHai. In other embodiments, the kit provides a combination with the NMDA receptor antagonist and the MAO inhibitor or GAPDHai mixed in one or more containers. The kits include a therapeutically effective dose of an agent for treating dementia-related conditions.

Oral Controlled-Release Formulations

As described above, the NMDA receptor antagonist, the MAO inhibitor or GAPDHai, or both agents may be provided in a controlled, extended release form. In one example, at least 50%, 90%, 95%, 96%, 97%, 98%, 99%, or even in excess of 99% of the NMDA receptor antagonist is provided in an extended release dosage form. A release profile, i.e., the extent of release of the NMDA receptor antagonist or the MAO inhibitor or GAPDHai over a desired time, may be conveniently determined for a given time by calculating the Cmax/Cmean for a desired time range. Thus, upon the administration to a subject (e.g., a mammal such as a human), the NMDA receptor antagonist has a Cmax/Cmean of approximately 2.5, 2, 1.5, or 1.0 approximately 1, 1.5, 2 hours to at least 6, 8, 9, 12, 18, 21, 24 hours following such administration. If desired, the release of the NMDA receptor antagonist may be monophasic or multiphasic (e.g., biphasic). Moreover, the MAO inhibitor or GAPDHai may be formulated as an extended release composition, having a Cₘₐₓ/Cₘₑₐₙ of approximately 2.5, 2, 1.5, or 1.0, approximately 1, 1.5, 2 hours to at least 6, 8, 9, 12, 18, 21, 24 hours following administration to a subject. One of ordinary skill in the art can prepare combinations with a desired release profile using the NMDA receptor antagonists and the MAO inhibitor or GAPDHai and formulation methods described below.

As shown in Table 2, the pharmacokinetic properties of each of the drugs of these classes varies from about 3 hours to 60 hours. Thus one aspect of this invention is to select suitable formulations to achieve nearly constant concentration profiles over an extended period (ideally from 8 to 24 hours) thereby maintaining both components in a constant ratio for optimal therapeutic benefits. Relative CRatios ranging from 0.4 to 2.5 from approximately 1, 1.5, 2 hours to at least 6, 8, 9, 12, 18, 21, 24 hours following administration to a subject are preferred. Formulations that deliver this constant, measurable profile are embodiments of the invention. Numerous ways exist for achieving the desired release profiles, as described below.

Suitable methods for preparing combinations in which the first component, second component, or both components are provided in extended release-formulations include those described in U.S. Pat. No. 4,606,909 (hereby incorporated by reference). This reference describes a controlled release multiple unit formulation in which a multiplicity of individually coated or microencapsulated units are made available upon disintegration of the formulation (e.g., pill or tablet) in the stomach of the animal (see, for example, column 3, line 26 through column 5, line 10 and column 6, line 29 through column 9, line 16). Each of these individually coated or microencapsulated units contains cross-sectionally substantially homogenous cores containing particles of a sparingly soluble active substance, the cores being coated with a coating that is substantially resistant to gastric conditions but which is erodable under the conditions prevailing in the small intestine.

The combination may alternatively be formulated using the methods disclosed in U.S. Pat. No. 4,769,027, for example. Accordingly, extended release formulations involve prills of pharmaceutically acceptable material (e.g., sugar/starch, salts, and waxes) may be coated with a water permeable polymeric matrix containing an NMDA receptor antagonist and next overcoated with a water-permeable film containing dispersed within it a water soluble particulate pore forming material.

One or both components of the combination may additionally be prepared as described in U.S. Pat. No. 4,897,268, involving a biocompatible, biodegradable microcapsule delivery system. Thus, the NMDA receptor antagonist may be formulated as a composition containing a blend of free-flowing spherical particles obtained by individually microencapsulating quantities of memantine, for example, in different copolymer excipients which biodegrade at different rates, therefore releasing memantine into the circulation at a predetermined rates. A quantity of these particles may be of such a copolymer excipient that the core active ingredient is released quickly after administration, and thereby delivers the active ingredient for an initial period. A second quantity of the particles is of such type excipient that delivery of the encapsulated ingredient begins as the first quantity's delivery begins to decline. A third quantity of ingredient may be encapsulated with a still different excipient which results in delivery beginning as the delivery of the second quantity beings to decline. The rate of delivery may be altered, for example, by varying the lactide/glycolide ratio in a poly(D,L-lactide-co-glycolide) encapsulation. Other polymers that may be used include polyacetal polymers, polyorthoesters, polyesteramides, polycaprolactone and copolymers thereof, polycarbonates, polyhydroxybuterate and copolymers thereof, polymaleamides, copolyaxalates and polysaccharides.

Alternatively, the combination may be prepared as described in U.S. Pat. No. 5,395,626 features a multilayered controlled release pharmaceutical dosage form. The dosage form contains a plurality of coated particles wherein each has multiple layers about a core containing an NMDA receptor antagonist and/or the MAOi or GAPDHain whereby the drug containing core and at least one other layer of drug active is overcoated with a controlled release barrier layer therefore providing at least two controlled releasing layers of a water soluble drug from the multilayered coated particle.

In some embodiments, the first component and second component of the combination described herein are provided within a single or separate pharmaceutical compositions. “Pharmaceutically or Pharmacologically Acceptable” includes molecular entities and compositions that do not produce an adverse, allergic or other untoward reaction when administered to an animal, or a human, as appropriate. “Pharmaceutically Acceptable Carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. “Pharmaceutically Acceptable Salts” include acid addition salts and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The preparation of pharmaceutical or pharmacological compositions are known to those of skill in the art in light of the present disclosure. General techniques for formulation and administration are found in “Remington: The Science and Practice of Pharmacy, Twentieth Edition,” Lippincott Williams & Wilkins, Philadelphia, Pa. Tablets, capsules, pills, powders, granules, dragees, gels, slurries, ointments, solutions suppositories, injections, inhalants and aerosols are examples of such formulations.

By way of example, extended release oral formulation can be prepared using additional methods known in the art. For example, a suitable extended release form of the either active pharmaceutical ingredient or both may be a matrix tablet composition. Suitable matrix forming materials include, for example, waxes (e.g., carnauba, bees wax, paraffin wax, ceresine, shellac wax, fatty acids, and fatty alcohols), oils, hardened oils or fats (e.g., hardened rapeseed oil, castor oil, beef tallow, palm dil, and soya bean oil), and polymers (e.g., hydroxypropyl cellulose, polyvinylpyrrolidone, hydroxypropyl methyl cellulose, and polyethylene glycol). Other suitable matrix tabletting materials are microcrystalline cellulose, powdered cellulose, hydroxypropyl cellulose, ethyl cellulose, with other carriers, and fillers. Tablets may also contain granulates, coated powders, or pellets. Tablets may also be multi-layered. Multi-layered tablets are especially preferred when the active ingredients have markedly different pharmacokinetic profiles. Optionally, the finished tablet may be coated or uncoated.

The coating composition typically contains an insoluble matrix polymer (approximately 15-85% by weight of the coating composition) and a water soluble material (e.g., approximately 15-85% by weight of the coating composition). Optionally an enteric polymer (approximately 1 to 99% by weight of the coating composition) may be used or included. Suitable water soluble materials include polymers such as polyethylene glycol, hydroxypropyl cellulose, hydroxypropyl methyl cellulose, polyvinylpyrrolidone, polyvinyl alcohol, and monomeric materials such as sugars (e.g., lactose, sucrose, fructose, mannitol and the like), salts (e.g., sodium chloride, potassium chloride and the like), organic acids (e.g., fumaric acid, succinic acid, lactic acid, and tartaric acid), and mixtures thereof. Suitable enteric polymers include hydroxypropyl methyl cellulose, acetate succinate, hydroxypropyl methyl cellulose, phthalate, polyvinyl acetate phthalate, cellulose acetate phthalate, cellulose acetate trimellitate, shellac, zein, and polymethacrylates containing carboxyl groups.

The coating composition may be plasticised according to the properties of the coating blend such as the glass transition temperature of the main component or mixture of components or the solvent used for applying the coating compositions. Suitable plasticisers may be added from 0 to 50% by weight of the coating composition and include, for example, diethyl phthalate, citrate esters, polyethylene glycol, glycerol, acetylated glycerides, acetylated citrate esters, dibutylsebacate, and castor oil. If desired, the coating composition may include a filler. The amount of the filler may be 1% to approximately 99% by weight based on the total weight of the coating composition and may be an insoluble material such as silicon dioxide, titanium dioxide, talc, kaolin, alumina, starch, powdered cellulose, MCC, or polacrilin potassium.

The coating composition may be applied as a solution or latex in organic solvents or aqueous solvents or mixtures thereof. If solutions are applied, the solvent may be present in amounts from approximate by 25-99% by weight based on the total weight of dissolved solids. Suitable solvents are water, lower alcohol, lower chlorinated hydrocarbons, ketones, or mixtures thereof. If latexes are applied, the solvent is present in amounts from approximately 25-97% by weight based on the quantity of polymeric material in the latex. The solvent may be predominantly water.

The pharmaceutical composition described herein may also include a carrier such as a solvent, dispersion media, coatings, antibacterial and antifuigal agents, isotonic and absorption delaying agents. The use of such media and agents for pharmaceutically active substances is well known in the art. Pharmaceutically acceptable salts can also be used in the composition, for example, mineral salts such as hydrochlorides, hydrobromides, phosphates, or sulfates, as well as the salts of organic acids such as acetates, proprionates, malonates, or benzoates. The composition may also contain liquids, such as water, saline, glycerol, and ethanol, as well as substances such as wetting agents, emulsifying agents, or pH buffering agents. Liposomes, such as those described in U.S. Pat. No. 5,422,120, WO 95/13796, WO 91/14445, or EP 524,968 B1, may also be used as a carrier.

Additional methods for making controlled release formulations are described in, e.g., U.S. Pat. Nos. 5,422,123, 5,601,845, 5,912,013, and 6,194,000, all of which are hereby incorporated by reference.

Non-Oral Formulations

Preparation for delivery in a transdermal patch can be performed using methods also known in the art, including those described generally in, e.g., U.S. Pat. Nos. 5,186,938 and 6,183,770, 4,861,800, and 4,284,444. A patch is a particularly useful embodiment in this case owing to first pass metabolism problems with many MAO inhibitors, including L-deprenyl. Patches can be made to control the release of skin-permeable active ingredients over a 12 hour, 24 hour, 3 day, and 7 day period. In one example, a 2-fold daily excess of an NMDA receptor antagonist is placed in a non-volatile fluid along with a MAO inhibitor or GAPDHai. Given the amount of the agents employed herein, a preferred release will be from 12 to 72 hours.

Transdermal preparations of this form will contain from 1% to 50% active ingredients. The compositions of the invention are provided in the form of a viscous, non-volatile liquid. Preferably, both members of the combination will have a skin penetration rate of at least 10⁻⁹ mole/cm²/hour. At least 5% of the active material will flux through the skin within a 24 hour period. The penetration through skin of specific formulations may be measures by standard methods in the art (for example, Franz et al., J. Invest. Derm. 64:194-195 (1975)).

In some embodiments, for example, the composition may be delivered intranasally to the cribriform plate rather than by inhalation to enable transfer of the active agents through the olfactory passages into the CNS and reducing the systemic administration. Devices commonly used for this route of administration are included in U.S. Pat. No. 6,715,485. Compositions delivered via this route may enable increased CNS dosing or reduced total body burden reducing systemic toxicity risks associated with certain drugs.

Preparation of a pharmaceutical composition for delivery in a subdermally implantable device can be performed using methods known in the art, such as those described in, e.g., U.S. Pat. Nos. 3,992,518; 5,660,848; and 5,756,115.

Indications Suitable for Treatment with the Combination

Any subject having or at risk of having dementia-related conditions, such as Parkinson's disease and Alzheimer's disease, may be treated using the combinations and methods described herein. Exemplary neuro-related conditions amenable to treatment according to the present invention are vascular dementia, senile dementia of the Alzheimer's type, minimal cognitive impairment, Lewy body dementia, Huntington's disease dementia, Pick's Disease, prion disease-related dementia, HIV-related dementia, frontotemporal dementia, hippocampal sclerosis-related dementia, encephalopathies-related dementias, and dementia related to neurodegenerative conditions, including demyelinating disease (e.g., multiple sclerosis (MS), progressive multifocal leukoencephalopathy (PML), disseminated necrotizing leukoencephalopathy (DNL), acute disseminated encephalomyelitis, Schilder disease, central pontine myelinolysis (CPM), radiation necrosis, Binswanger disease (SAE), Guillain-Barre Syndrome, leukodystrophy, acute disseminated encephalomyelitis (ADEM), acute transverse myelitis, acute viral encephalitis, adrenoleukodystrophy (ALD), adrenomyeloneuropathy, AIDS-vacuolar myelopathy, experimental autoimmune encephalomyelitis (EAE), experimental autoimmune neuritis (EAN), HTLV-associated myelopathy, Leber's hereditary optic atrophy, subacute sclerosing panencephalitis, and tropical spastic paraparesis), Parkinson's disease, Alzheimer's disease, prion-related diseases, psychiatric disorders (e.g., mood, depression, anxiety, attention deficit disorder, autism, behavior/conduct disorders, dissociative disorders, eating disorders, fetal alcohol syndrome, learning disabilities, mental retardation, mood disorders, speech and language, substance abuse, suicide, Tourette's disorder, and post traumatic stress syndrome), seizures and convulsive disorders (e.g., epilepsy), pain (e.g., central and peripheral, including acute, chronic and neuropathic), migraine and acute neurodegenerative disorders like trauma and stroke. Any of these conditions may be treated using the methods and compositions described herein.

Using the Combinations

Treatment of a subject with the combination may be monitored using methods known in the art. The efficacy of treatment using the combination is preferably evaluated by examining the subject's symptoms in a quantitative way, e.g., by noting a decrease in the frequency of relapses, or an increase in the time for sustained worsening of symptoms. In a successful treatment, the subject's status will have improved (i.e., frequency of relapses will have decreased, or the time to sustained progression will have increased).

### EXAMPLES

The invention will be illustrated in the following non-limiting examples.

### Example 1

### In Vitro Method for Determining Optimal Synergy

We employ the protocol described in Parsons (Parsons, C G et al. Neuropharmacology 38: 85-108, 1999) and Weller (Weller et al., Brain Research 613: 143-148, 1993) for this purpose. Briefly, 13-14-day primary cultures of embryonic rat cortices are seeded onto 11 mm wells Cultures are kept at 37° C. in 95% air/5% CO₂. In order to decrease the number of non-neuronal cells, the antimitotic cytosine arabinoside (araC) is used at 10⁻⁶ M starting on the third day of culture during 3 days. Just prior to glutamate treatment, the culture medium is replaced with HEPES-buffered control salt solution pH 7.4 (HCSS). Cells are incubated with 1 mM glutamate plus test compound or the reference compound, MK-801. After a 10 min period of incubation at room temperature, this solution is removed and replaced by serum-free MEM with plus test compound or the reference compound, and the cells are re-incubated at 37° C. for 24 h under standard conditions. After morphological examination of the cells, the supernatants from the control and treated cultures are harvested and analysed for LDH activity.

A dose ranging study is performed first on memantine to determine the ED50, expected in the range of 1-10 um. The ED50 for selegiline is determined in a similar manner. An isobolic experiment ensues where the drugs are combined in fractions of their EDXXs to add up to ED100 (i.e., ED50:ED50, ED25:ed75, etc.). The plot of the data is constructed. If the experiment point lie below the straight line between the ED50 points on the graph, the combination is synergistic, on the line is additive, and above the line is inhibitory. The point of maximum synergistic deviation from the isobolic line is the optimal ratio. This is the optimal steady state ratio (C_{ratio,ss}) and is adjusted based upon the components half-life.

### Example 2

### In Vivo Method for Determining Optimal Steady-State Concentration Ratio (Cratio,ss)

The optimal steady state concentration is determined with the MPTP model of PD (Fredriksson A, Danysz W, Quack G and Archer T. 2001. J. Neural Transm 108: 167-187), but any relevant CNS model may be used for this purpose. Briefly, mice are injected sc with MPTP, 80 mg/kg every 24 hrs for 8-9 weeks to establish stable Parkinsonian syndrome. Animals are treated with L-dopa, 20 mg/kg sc, everyday for 5 days/week for 5 weeks. L-dopa-tolerant MPTP mice are administered test compound or saline before being placed in an activity test chamber. The mice are then injected with L-dopa or saline and motor activity is scored over 3 hours.

A dose ranging study is performed first on memantine to determine the ED 50, expected in the range of 1-10 um. The ED50 for 1-deprenyl is determined in a similar manner. An isobolic experiment ensues where the drugs are combined in fractions of their EDXXs to add up to ED100 (i.e., ED50:ED50, ED25:ED75, etc.). The plot of the data is constructed. The experiment points that lie below the straight line between the ED50 points on the graph are indicative of synergy, on the line is additive, and above the line is inhibitory. The point of maximum synergistic deviation from the isobolic line is the optimal ratio. This is the optimal steady state ratio (C_{ratio,ss}) and is adjusted based upon the components half-life.

### Example 3

### Combinations of an NMDA Receptor Antagonist and an MOA Inhibitor

Representative combination ranges are provided below for compositions of the invention.

Adult Dosage for Combination Therapy

| NMDA drug | MAO inhibitor or GAPDHai (mg/day) | | |
|---|---|---|---|
| mg/day | LDeprenyl/Selegiline | Desmethyl Deprenyl | Rasagiline |
| Memantine/ | 0.5-10 | 0.5-10 | 0.5-2.0 |
| 2.5-40 | | | |
| Amantadine/ | 0.5-10 | 0.5-10 | 0.5-2.0 |
| 50-300 | | | |
| Rimantadine/ | 0.5-10 | 0.5-10 | 0.5-2.0 |
| 50-200 | | | |

### Example 4

### Release Profile of Memantine and L-deprenyl Combination

Release proportions are shown in the tables below. The cumulative fraction is the amount of drug substance released from the formulation matrix to the serum or gut environment (e.g., U.S. Pat. No. 4,839,177).

| | MEMANTINE | L-DEPRENYL |
|---|---|---|
| | T½ = 60 hrs | T½ = 1-4 hrs |
| Time | cum. fraction A | cum. fraction B |
| 0.5 | 0.2 | 0.2 |
| 2 | 0.3 | 0.3 |
| 4 | 0.4 | 0.4 |
| 8 | 0.5 | 0.5 |
| 12 | 0.6 | 0.6 |
| 16 | 0.7 | 0.7 |
| 20 | 0.8 | 0.8 |
| 24 | 0.9 | 0.9 |

| | MEMANTINE | L-DEPRENYL |
|---|---|---|
| | T½ = 60 hrs | T½ = 1-4 hrs |
| Time | cum. fraction A | cum. fraction B |
| 0.5 | 0.2 | 0.30 |
| 2 | 0.3 | 0.40 |
| 4 | 0.4 | 0.50 |
| 8 | 0.5 | 0.60 |
| 12 | 0.6 | 0.70 |
| 16 | 0.7 | 0.80 |
| 20 | 0.8 | 0.90 |
| 24 | 0.9 | 0.99 |

### Example 5

### Tablet Containing a Combination of Memantine and L-DEPRENYL

A pulsatile release dosage form for administration of memantine and L-deprenyl is prepared as three individual compartments. Three individual compressed tablets, each having a different release profile, followed by (2) encapsulating the three tablets into a gelatin capsule and then closing and sealing the capsule. The components of the three tablets are as follows.

| | | |
|---|---|---|
| Component | Function | Amount per tablet |

TABLET 1 (IMMEDIATE RELEASE):

| | | |
|---|---|---|
| Memantine | Active agent | 8 mg |
| L-deprenyl | Active agent | 5 mg |
| Dicalcium phosphate dihydrate | Diluent | 26.6 mg |
| Microcrystalline cellulose | Diluent | 26.6 mg |
| Sodium starch glycolate | Disintegrant | 1.2 mg |
| Magnesium Stearate | Lubricant | 0.6 mg |

TABLET 2 (RELEASE DELAYED 3-5 HOURS FOLLOWING
ADMINISTRATION):

| | | |
|---|---|---|
| Memantine | Active agent | 8 mg |
| L-deprenyl | Active agent | 5 mg |
| Dicalcium phosphate dihydrate | Diluent | 26.6 mg |
| Microcrystalline cellulose | Diluent | 26.6 mg |
| Sodium starch glycolate | Disintegrant | 1.2 mg |
| Magnesium Stearate | Lubricant | 0.6 mg |
| Eudragit RS30D | Delayed release | 4.76 mg |
| | coating material | |
| Talc | Coating component | 3.3 mg |
| Triethyl citrate | Coating component | 0.95 mg |

TABLET 3 (RELEASE DELAYED 7-9 HOURS FOLLOWING
ADMINISTRATION):

| | | |
|---|---|---|
| Memantine | Active agent | 2.5 mg |
| L-deprenyl | Active agent | 5 mg |
| Dicalcium phosphate dihydrate | Diluent | 26.6 mg |
| Microcrystalline cellulose | Diluent | 26.6 mg |
| Sodium starch glycolate | Disintegrant | 1.2 mg |
| Magnesium Stearate | Lubricant | 0.6 mg |
| Eudragit RS30D | Delayed release | 6.34 mg |
| | coating material | |
| Talc | Coating component | 4.4 mg |
| Triethyl citrate | Coating component | 1.27 mg |

The tablets are prepared by wet granulation of the individual drug particles and other core components as may be done using a fluid-bed granulator, or are prepared by direct compression of the admixture of components. Tablet 1 is an immediate release dosage form, releasing the active agents within 1-2 hours following administration. Tablets 2 and 3 are coated with the delayed release coating material as may be carried out using conventional coating techniques such as spray-coating or the like. The specific components listed in the above tables may be replaced with other functionally equivalent components, e.g., diluents, binders, lubricants, fillers, coatings, and the like.

Oral administration of the capsule to a patient will result in a release profile having three pulses, with initial release of the memantine and L-deprenyl from the first tablet being substantially immediate, release of the memantine and L-deprenyl from the second tablet occurring 3-5 hours following administration, and release of the memantine and L-deprenyl from the third tablet occurring 7-9 hours following administration. The effective profile will be nearly linear over the range, leading to concentration profiles.

### Example 7

### Beads Containing a Combination of Memantine and L-Deprenyl

The method of Example 6 is repeated, except that drug-containing beads are used in place of tablets. A first fraction of beads is prepared by coating an inert support material such as lactose with the drug which provides the first (immediate release) pulse. A second fraction of beads is prepared by coating immediate release beads with an amount of enteric coating material sufficient to provide a drug release-free period of 3-7 hours. A third fraction of beads is prepared by coating immediate release beads having half the methylphenidate dose of the first fraction of beads with a greater amount of enteric coating material, sufficient to provide a drug release-free period of 7-12 hours. The three groups of beads may be encapsulated as in Example 3, or compressed, in the presence of a cushioning agent, into a single pulsatile release tablet. The resulting release profile is nearly linear over a 12 hour range.

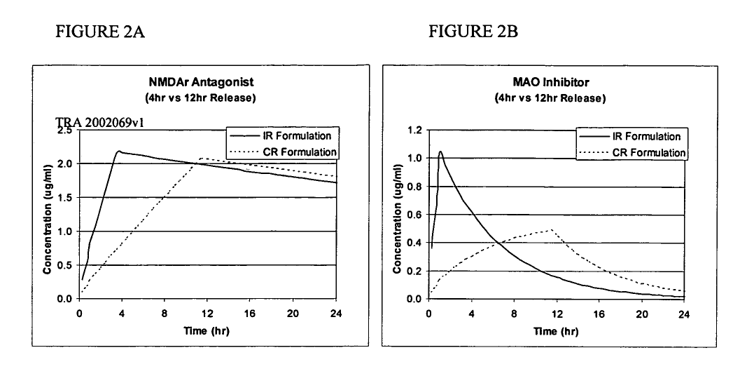

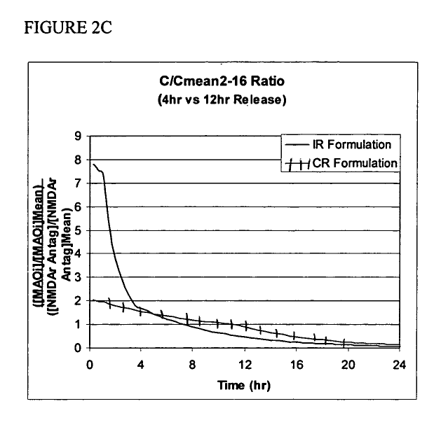

Alternatively, three groups of drug particles may be provided and coated as above, in lieu of the drug-coated lactose beads. A exemplary release profile is shown in FIGS. 2A-2C, a series of graphs showing the release profiles and Cratio for controlled release combination product. This product will maintain a nearly constant ratio of the two components, ranging from 2 times the average CRatio (set=1) to 0.5 as the time ranges from 2 to 16 hours.

In addition to achieving the desired release profile, this combination formulation will exhibit preferred concentration increase of 0.2 v. 0.5.

| | |
|---|---|
| IR | CR |

NMDAr Antag

| | | |
|---|---|---|
| dC/dT (4 hr) | 0.54 | 0.20 |
| Cmax/Cmean2-16 | 1.10 | 1.38 |

MAOi

| | | |
|---|---|---|
| dC/dT (1 hr) | 1.04 | 0.13 |
| Cmax/Cmean2-16 | 3.11 | 1.35 |

### Example 8

### Patch Providing Extended Release of Memantine and 1-deprenyl

As described above, extended release formulations of an NMDA antagonist may be formulated for topical administration. Memantine transdermal patch formulations may be prepared as described, for example, in U.S. Pat. Nos. 6,770,295 and 6,746,689, hereby incorporated by reference.

For the preparation of a drug-in-adhesive acrylate, 5 g of memantine and 4 g of L-deprenyl will be dissolved in 11 g of ethanol and is added to 20 g of Durotak 387-2287 (National Starch & Chemical, U.S.A.). The drug gel is coated onto a backing membrane (Scotchpak 1012; 3M Corp., U.S.A.) using a coating equipment (e.g., RK Print Coat Instr. Ltd, Type KCC 202 control coater). The wet layer thickness is 400 μm. The laminate is dried for 20 minutes at room temperature and then for 30 minutes at 40° C. A polyester release liner is laminated onto the dried drug gel. The sheet is cut into patches and stored at 2-8° C. until use (packed in pouches). The concentration of memantine in the patches will range between 5.6 and 8 mg/cm², while the L-deprenyl will range between 2.8 and 6.5 mg/cm².

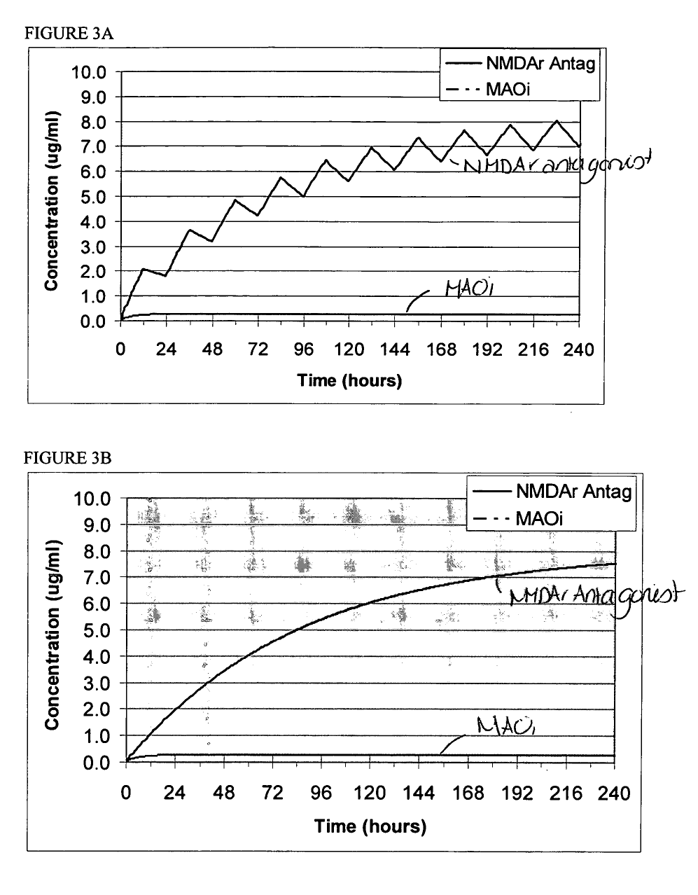

FIGS. 3A and 3B are graphs compares the anticipated 12 hour controlled release (example 7) with the anticipated 24 hour of the current example. These graphs indicate the advantage of nearly continuous infusion of the components, and the importance of establishing the correct steady-state ratio (Cratio,ss) and then modifying the dosage form concentrations to achieve the optimal.

### Example 9

### Patch Providing Extended Release of Amantadine and 1-deprenyl

A patch allowing the extended release of amantadine and sele may be prepared as follows. The matrix patch is composed of 1 mm thick polyolefin foam (as an occlusive backing) coated with an acrylate matrix that includes a mixture of amantadine, 1-deprenyl and an intradermal-penetration agent in an acrylate polymer. The matrix is prepared by mixing amantadine (20 weight percent); 1-dperenyl (20 weight percent); acrylate polymer (Durotak.RTM. 387-2052, 75 weight percent); intradermal-penetration agent; aluminumacetylacetonate (Al(ACAC)₃, 0.4 weight percent, as a crosslinker); and ethanol until homogeneous. The homogeneous mixture is then coated on polyolefin foil with a hand-coater machine to an average thickness of about 270 μm. The coated foil is dried for about one hour at about 50° C. to evaporate the ethanol. The resulting patch weighs approximately 50 g/m² dry.

### Equivalents

Those skilled in the art will recognize, or be able to ascertain using no more than routine experimentation, numerous equivalents to the specific procedures described herein. Such equivalents are considered to be within the scope of the present invention and are covered by the following claims. Various substitutions, alterations, and modifications may be made to the invention without departing from the spirit and scope of the invention as defined by the claims. Other aspects, advantages, and modifications are within the scope of the invention. The contents of all references, issued patents, and published patent applications cited throughout this application are hereby fully incorporated by reference. The appropriate components, processes, and methods of those patents, applications and other documents may be selected for the present invention and embodiments thereof.

1. A pharmaceutical composition comprising:
(a) an NMDA receptor antagonist;
(b) a second agent, wherein said agent is a monoamine oxidase (MAO) inhibitor or a GADPH inhibitor; and
(c) a pharmaceutically acceptable carrier
wherein said NMDA receptor antagonist, said second agent, or both are in an extended release dosage form.

2. The pharmaceutical composition of claim 1, wherein said NMDA receptor antagonist is provided in an extended release dosage form.

3. The pharmaceutical composition of claim 2, wherein said NMDA receptor antagonist has a Cₘₐₓ/Cₘₑₐₙ of approximately 2 or less, approximately 2 hours to at least 6 hours after said NMDA receptor antagonist is introduced into a subject.

4. The pharmaceutical composition of claim 1, wherein the relative Cratio of said NMDA receptor antagonist and said second agent is 0.4-2.5.

5. The pharmaceutical composition of claim 2, wherein at least 50% of said NMDA receptor antagonist in said pharmaceutical composition is provided in an extended release dosage form.

6. The pharmaceutical composition of claim 5, wherein 95% of said NMDA receptor antagonist in said pharmaceutical composition is provided in an extended release dosage form.

7. The pharmaceutical composition of claim 6, wherein essentially all of said NMDA receptor antagonist in said pharmaceutical composition is provided in an extended release dosage form.

8. The pharmaceutical composition of claim 2, wherein at least 99% of said NMDA receptor antagonist remains in said extended dosage form one hour following introduction of said pharmaceutical composition into a subject.

9. The pharmaceutical composition of claim 1, wherein said second agent is provided in an extended release dosage form.

10. The pharmaceutical composition of claim 9, wherein said second agent has a Cₘₐₓ/Cₘₑₐₙ of approximately 2 or less, approximately 2 hours to at least 6 hours after said second agent is introduced into a subject.

11. The pharmaceutical composition of claim 10, wherein said second agent has a Cₘₐₓ/Cₘₑₐₙ of approximately 2 or less, approximately 2 hours to at least 12 hours after said second agent is introduced into a subject.

12. The pharmaceutical composition of claim 11, wherein said NMDA receptor antagonist has a Cₘₐₓ/Cₘₑₐₙ of approximately 2 or less, approximately 2 hours to at least 6 hours after said NMDA receptor antagonist is introduced into a subject.

13. The pharmaceutical composition of claim 1, wherein said NMDA receptor antagonist and said second agent are both provided in an extended release dosage form.

14. The pharmaceutical composition of claim 1, wherein said NMDA receptor antagonist is an aminoadamantine derivative.

15. The pharmaceutical composition of claim 14, wherein said aminoadamantine derivative is memantine (1-amino-3,5-dimethyladamantane), rimantadine (1-(1-aminoethyl)adamantane), or amantadine (1-amino-adamantane).

16. The pharmaceutical composition of claim 15, wherein said aminoadamantine derivative is memantine (1-amino-3,5-dimethyladamantane).

17. The pharmaceutical composition of claim 1, wherein said second agent is selegiline, rasagaline, desmethyldeprenyl, CGP3466, phenelzine, or tranycypromine.

18. The pharmaceutical composition of claim 17, wherein said second agent is selegiline.

19. The pharmaceutical composition of claim 1, wherein said NMDA receptor antagonist is memantine and said second agent is selegiline.

20. The pharmaceutical composition of claim 1, wherein said pharmaceutical composition is formulated for oral, intravenous, subtopical transepithelial, subdermal, or inhalation delivery.

21. The pharmaceutical composition of claim 20, wherein said pharmaceutical composition is formulated as a suspension, capsule, tablet, suppository, lotion, or patch.

22. The pharmaceutical composition of claim 1, wherein said NMDA receptor antagonist and said second agent are provided in a unit dosage form.

23. The pharmaceutical composition of claim 1, wherein the amount of said NMDA receptor antagonist in said pharmaceutical composition is less than the amount of NMDA receptor antagonist required in a unit dose to obtain the same therapeutic effect for treating CNS-related condition when the NMDA receptor antagonist is administered in the absence of said second agent.

24. The pharmaceutical composition of claim 1, wherein the amount of said second agent in said pharmaceutical composition is less than the amount of said second agent required in a unit dose to obtain the same therapeutic effect for treating CNS-related condition when said second agent is administered in the absence of the NMDA receptor antagonist.

25. The pharmaceutical composition of claim 1, wherein said NMDA receptor antagonist is present in said pharmaceutical composition at a dose that would be toxic to a human subject if said NMDA receptor antagonist were administered to said subject in the absence of said second agent.

26. The pharmaceutical composition of claim 1, wherein said second agent is present in said pharmaceutical composition at a dose that would be toxic to a human subject if said second agent were administered to said subject in the absence of said second agent.

27. A method of treating a CNS-related condition comprising administering to a subject in need thereof a therapeutically effective amount of a combination comprising an NMDA receptor antagonist and a second agent, wherein said second agent is a MAO inhibitor or a GADPH inhibitor.

28. The method of claim 27, wherein said NMDA receptor antagonist is provided in an extended release dosage form.

29. The method of claim 28, wherein said NMDA receptor antagonist has a Cₘₐₓ/Cₘₑₐₙ of approximately 2 or less, approximately 2 hours to at least 6 hours after said NMDA receptor antagonist is introduced into a subject.

30. The method of claim 29, wherein said NMDA receptor antagonist has a Cₘₐₓ/Cₘₑₐₙ of approximately 2 or less approximately 2 hours to at least 12 hours after said NMDA receptor antagonist is introduced into a subject.

31. The method of claim 27, wherein at least 50% of said NMDA receptor antagonist in said pharmaceutical composition is provided in an extended release dosage form.

32. The method of claim 31, wherein 95% of said NMDA receptor antagonist in said pharmaceutical composition is provided in an extended release dosage form.

33. The method of claim 32, wherein essentially all of said NMDA receptor antagonist in said pharmaceutical composition is provided in an extended release dosage form.

34. The method of claim 31, wherein at least 99% of said NMDA receptor antagonist is remains in said extended dosage form one hour following introduction of said pharmaceutical composition into a subject.

35. The method of claim 27, wherein said second agent is provided in an extended release dosage form.

36. The method of claim 25, wherein said second agent has a Cₘₐₓ/Cₘₑₐₙ of approximately 2 or less, approximately 2 hours to at least 6 hours after said second agent is introduced into a subject.

37. The method of claim 36, wherein said second agent has a Cₘₐₓ/Cₘₑₐₙ of approximately 2 or less, approximately 2 hours to at least 12 hours after said second agent is introduced into a subject.

38. The method of claim 27, wherein said NMDA receptor antagonist has a Cₘₐₓ/Cₘₑₐₙ of approximately 2 or less, approximately 2 hours to at least 6 hours after said NMDA receptor antagonist is introduced into a subject.

39. The method of claim 27, wherein said NMDA receptor antagonist is a low affinity NMDA receptor antagonist.

40. The method of claim 27, wherein said NMDA receptor antagonist is an aminoadamantine derivative.

41. The method of claim 40, wherein said aminoadamantine derivative is memantine (1-amino-3,5-dimethyladamantane), rimantadine (1-(1-aminoethyl)adamantane), or amantadine (1-amino-adamantane).

42. The method of claim 41, wherein said aminoadamantine derivative is memantine (1-amino-3,5-dimethyladamantane).

43. The method of claim 27, wherein said second agent is selegiline, rasagaline, desmethyldeprenyl, CGP3466, phenelzine or tranycypromine.

44. The method of claim 27, wherein said NMDA receptor antagonist is memantine and said second agent is selegiline.

45. The method of claim 27, wherein said CNS-related condition is Parkinson's disease, Alzheimer's disease, or multiple sclerosis.

46. The method of claim 27, wherein said NMDA receptor antagonist is delivered orally, intravenouslly, subdermally, or by inhalation.

47. The method of claim 27, wherein said second agent is delivered orally, intravenouslly, subdermally, or by inhalation.

48. The method of claim 27, wherein said NMDA receptor antagonist and said second agent are administered simultaneously.

49. The method of claim 27, wherein said NMDA antagonist and said second agent are administered as a single composition

50. The method of claim 27, wherein said NMDA antagonist and said second agent are administered sequentially.

51. The method of claim 27, wherein said NMDA receptor antagonist and said second agent are administered within 24 hours of each other.

52. The method claim 27, wherein said NMDA receptor antagonist and said second agent are administered by the same route of administration.

53. The method of claim 27, wherein said NMDA receptor antagonist and said second agent are administered by different routes of administration.

54. The method of claim 27, wherein said NMDA receptor antagonist, said second agent, or both are administered to said subject once a day.

55. The method of claim 27, wherein said NMDA receptor antagonist, said second agent, or both are administered to said subject every three days.

56. The method of claim 27, wherein said subject is a human.
